(19) United States
(12) Patent Application Publication
(10) Pub. No.: US 2021/0275772 A1
(43) Pub. Date: Sep. 9, 2021

(54) Sting Alleviation Apparatus

(71) Applicants: Paul Hemingway, Canyon Country, CA (US); Chyenne Hemingway, Canyon County, CA (US)

(72) Inventors: Paul Hemingway, Canyon Country, CA (US); Chyenne Hemingway, Canyon County, CA (US)

(21) Appl. No.: 16/808,043

(22) Filed: Mar. 3, 2020

(51) Int. Cl. A61M 19/00; A61M 11/02; A61B 17/32; A61K 31/167; A61K 31/14

(52) U.S. Cl. CPC A61M 19/00; A61M 11/02; A61B 17/32; A61M 2205/586; A61K 31/14; A61B 2017/320008; A61M 2210/04; A61K 31/167

(57) ABSTRACT

A sting alleviation apparatus for controlling the reaction to a bee sting and removing a bee stinger includes a bee sting alleviation solution comprising at least 80% dimethyl ether, lidocaine, and benzalkonium chloride. The bee sting alleviation solution is contained in a sting alleviation apparatus comprising a can body having a spray head depressibly coupled to a can top. A cap is coupled to the can body. The cap has a cap sidewall and a cap top. A lower edge of the cap sidewall is selectively engageable with a lower rim of the can top to cover the can top and the spray head. A scraper is coupled to the cap sidewall adjacent the cap top. A distal edge of the scraper is serrated and configured to remove a bee stinger from skin.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to bee sting treatments and more particularly pertains to a new bee sting treatment for controlling the reaction to a bee sting and removing a bee stinger.

### (2) Description of Related Art Including Information Disclosed Under 37 CFR 1.97 and 1.98

The prior art relates to bee sting treatments.

### BRIEF SUMMARY OF THE INVENTION

An embodiment of the disclosure meets the needs presented above by generally comprising a can body having a can bottom, a can sidewall, a dome-shaped can top, and a spray head depressibly coupled to the can top. The spray head releases a pressurized content held within the can body through a nozzle when depressed. A cap is coupled to the can body. The cap has a cap sidewall and a cap top. A lower edge of the cap sidewall is selectively engageable with a lower rim of the can top to cover the can top and the spray head. A scraper is coupled to the cap sidewall adjacent the cap top. A distal edge of the scraper is serrated and configured to remove a bee stinger from skin.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a sting alleviation apparatus according to an embodiment of the disclosure.

FIG. 2 is a top plan view of an embodiment of the disclosure.

FIG. 3 is a bottom plan view of an embodiment of the disclosure.

FIG. 4 is a side elevation detail view of an embodiment of the disclosure.

FIG. 5 is a block diagram of an embodiment of the disclosure.

### DETAILED DESCRIPTION OF THE INVENTION

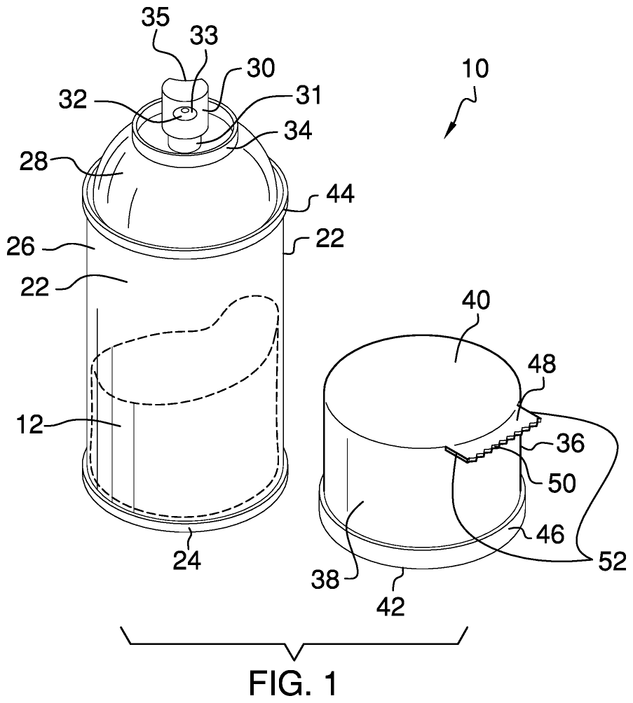

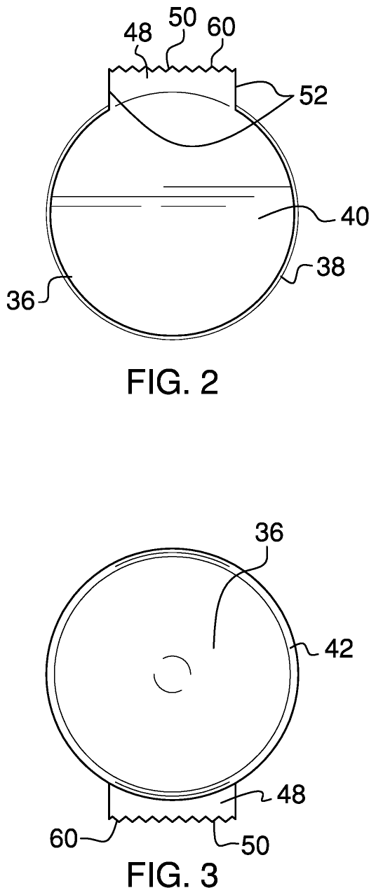

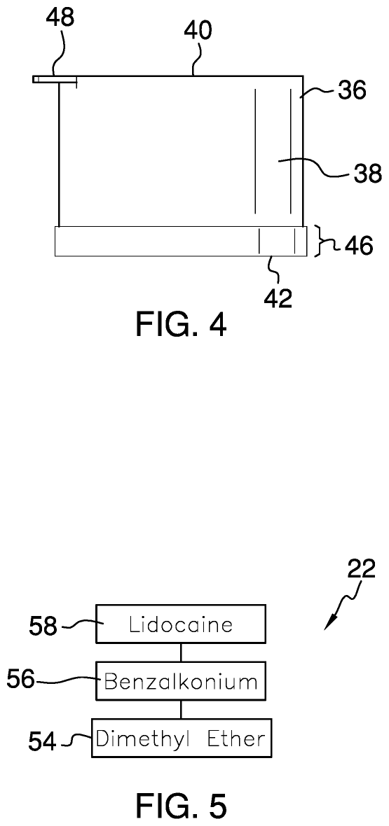

With reference now to the drawings, and in particular to FIGS. 1 through 5 thereof, a new bee sting treatment embodying the principles and concepts of an embodiment of the disclosure and generally designated by the reference numeral 10 will be described.

As best illustrated in FIGS. 1 through 5, the sting alleviation apparatus 10 generally comprises a can body 20 containing a bee sting alleviation solution 22. The can body 20 has a can bottom 24, a can sidewall 26, a dome-shaped can top 28, and a spray head 30 depressibly coupled to the can top 28. The spray head 30 may be cylindrical with a thinner neck portion 31 closer to the can top 28. The spray head 30 releases the bee sting alleviation solution 22 through a nozzle 32 when depressed. The nozzle 32 may be recessed within a conical aperture 33 of the spray head 30. The spray head 30 may also have a rounded finger cutout 35 opposite the nozzle 32 for ergonomic purposes and to guide a user to easily know the direction to spray the bee sting alleviation solution 22. The can body 20 may come in a variety of sizes ranging from a pocketsize version to a full size can dimensioned to the size of a standard spray paint can. The can body 20 may have a round collar 34 coupled to the can top 28 around the spray head 30 to prevent drips from running down the can body 20.

A cap 36 is coupled to the can body 20. The cap 36 has a cap sidewall 38 and a cap top 40. A lower edge 42 of the cap sidewall is selectively engageable with a lower rim 44 of the can top to cover the can top 28 and the spray head 30. The cap sidewall 38 may have a wide portion 46 adjacent the lower edge 42 to accommodate the lower rim 44 and engage with a press fit to maintain the cap 36 in place. A scraper 48 is coupled to the cap sidewall 38 and may lie coplanar with the cap top 40. The scraper 48 has a serrated distal edge 50 extending perpendicularly between a pair of parallel side edges 52. The distal edge 50 is configured to remove a bee stinger from skin. The side edges 52 protrude past the wide portion 46 to ensure the distal edge 50 can easily be placed in contact with the user's skin without also contacting the cap sidewall 38. The scraper 48 may occupy 30% to 60% of a circumference of the cap top 40 between the side edges 52 to create a sufficiently wide distal edge 50 to scrape the skin.

The bee sting alleviation solution 22 comprises at least 80% dimethyl ether 54, 5% to 15% lidocaine 56, and 5% to 15% benzalkonium chloride 58. The bee sting alleviation solution 22 may comprise equal parts lidocaine 56 and benzalkonium chloride 58. Further, the bee sting alleviation solution 22 may comprise a ratio of eight parts dimethyl ether 54 to one part lidocaine 56 and one part benzalkonium chloride 58. For example, a four ounce sample would contain 3.2 ounces dimethyl ether 54, 0.4 ounces lidocaine 56, and 0.4 ounces benzalkonium chloride 58.

In use, the cap 36 is secured either independently or while engaged with the can body 20 to manipulate the scraper 48 to remove the bee stinger after being stung. The serrated distal edge 50 can be moved back and forth over the sting or an individual tooth 60 of the distal edge 50 may be used to lift the bee stinger. The spray head 30 is then depressed to apply the bee sting alleviation solution 22 onto the area of the sting to prevent swelling and itching.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sting alleviation apparatus comprising:
a can body, the can body having a can bottom, a can sidewall, a dome-shaped can top, and a spray head depressibly coupled to the can top, the spray head releasing a pressurized content held within the can body through a nozzle when depressed;
a cap coupled to the can body, the cap having a cap sidewall and a cap top, a lower edge of the cap sidewall being selectively engageable with a lower rim of the can top to cover the can top and the spray head; and
a scraper coupled to the cap, the scraper being coupled to the cap sidewall adjacent the cap top, a distal edge of the scraper being serrated, the scraper being configured to remove a bee stinger from skin.

2. The sting alleviation apparatus of claim 1 further comprising the scraper being coplanar with the can top.

3. The sting alleviation apparatus of claim 2 further comprising the distal edge extending perpendicularly between a pair of parallel side edges.

4. The sting alleviation apparatus of claim 1 further comprising the cap sidewall having a wide portion adjacent the lower edge.

5. The sting alleviation apparatus of claim 1 further comprising a round collar coupled to the can top around the spray head.

5. The sting alleviation apparatus of claim 1 further comprising the pressurized content being a bee sting alleviation solution comprising at least 80% dimethyl ether, 5% to 15% lidocaine, and 5% to 15% benzalkonium chloride.

6. The bee sting alleviation solution of claim 5 further comprising there being equal parts lidocaine and benzalkonium chloride.

8. A sting alleviation apparatus comprising:
a can body, the can body having a can bottom, a can sidewall, a dome-shaped can top, and a spray head depressibly coupled to the can top, the spray head releasing a pressurized content held within the can body through a nozzle when depressed, the can body having a round collar coupled to the can top around the spray head;
a cap coupled to the can body, the cap having a cap sidewall and a cap top, a lower edge of the cap sidewall being selectively engageable with a lower rim of the can top to cover the can top and the spray head, the cap sidewall having a wide portion adjacent the lower edge; and
a scraper coupled to the cap, the scraper being coupled to the cap sidewall and coplanar with the cap top, the scraper having a serrated distal edge extending perpendicularly between a pair of parallel side edges, the scraper being configured to remove a bee stinger from skin.

9. A sting alleviation apparatus comprising:
a can body, the can body having a can bottom, a can sidewall, a dome-shaped can top, and a spray head depressibly coupled to the can top, the spray head releasing a pressurized content held within the can body through a nozzle when depressed, the can body having a round collar coupled to the can top around the spray head, the pressurized content being a bee sting alleviation solution comprising 80% dimethyl ether, 10% lidocaine, and 10% benzalkonium chloride;
a cap coupled to the can body, the cap having a cap sidewall and a cap top, a lower edge of the cap sidewall being selectively engageable with a lower rim of the can top to cover the can top and the spray head, the cap sidewall having a wide portion adjacent the lower edge; and
a scraper coupled to the cap, the scraper being coupled to the cap sidewall and coplanar with the cap top, the scraper having a serrated distal edge extending perpendicularly between a pair of parallel side edges, the scraper being configured to remove a bee stinger from skin.
